(19) United States
(12) Patent Application Publication
(10) Pub. No.: US 2015/0299805 A1
(43) Pub. Date: Oct. 22, 2015

(54) NUCLEIC ACID DETECTION ASSAY

(71) Applicant: Amos Douvdevani, Beer-Sheva (IL)

(72) Inventor: Amos Douvdevani, Beer-Sheva (IL)

(73) Assignees: BEN GURION UNIVERSITY OF THE NEGEV RESEARCH AND DEVELOPMENT AUTHORITY, BEER-SHEVA (IL); MOR RESEARCH APPLICATIONS LTD., TEL AVIV (IL)

(21) Appl. No.: 14/593,347

(22) Filed: Jan. 9, 2015

Related U.S. Application Data
(63) Continuation of application No. 13/659,439, filed on Oct. 24, 2012, now abandoned.
Application No. 13/659,439 is a continuation-in-part of application No. 12/667,186, filed on May 6, 2010, now abandoned.
(60) Provisional application No. 60/996,900, filed on Dec. 10, 2007.

(51) Int. Cl. C12Q 1/68; G01N 21/64; G06F 19/00; G01N 33/58; G06F 19/24

(52) U.S. Cl. CPC C12Q 1/6886; G01N 33/582; G06F 19/24; G06F 19/345; G01N 21/64

(57) ABSTRACT

This invention is directed, inter alia, to methods and kits for rapid, easy and cost-effective methods of breast cancer prediction and diagnosis in inter alia, blood samples.

### CROSS REFERENCE TO RELATED APPLICATIONS

This application is a Continuation of U.S. patent application Ser. No. 13/659,439, filed Oct. 24, 2012, which is a Continuation-In-Part application of U.S. patent application Ser. No. 12/667,186, filed May 6, 2010, which is a '371 application of PCT International Application Number PCT/IL2008/001584, filed Dec. 4, 2008, which claims the benefit of U.S. Provisional Application No. 60/996,900, filed Dec. 10, 2007, all of which are fully incorporated herein by reference.

### BACKGROUND OF THE INVENTION

There is an increase in the amount of free DNA in the blood that is correlated with cell death, as a function of tissue injury or inflammatory responses. An increase in free DNA in the blood as a result of many diseases has been seen, including autoimmune disease, stroke, cancer and cardiovascular disease. Free DNA levels have been considered to be a telling prognostic for these and other diseases, yet the methodology to quantitatively assess free circulating DNA levels is expensive and time consuming.

There is an increase in the amount of free DNA in the blood that is correlated with cell death, as a function of tissue injury or inflammatory responses, or other diseases. For example, one characteristic property of cancer and other cell proliferative diseases is an increased amount of free floating, circulating DNA in blood and/or serum. Cell death caused by for example toxic doses of bacterial lipopolysaccharide, and toxic chemicals triggers the release of products of chromatin catabolism, particularly of DNA into extracellular spaces, which may also be detected by quantification of serum or blood DNA levels. An increase in free DNA in the blood as a result of many diseases has been seen, including autoimmune diseases, stroke, cancer and cardiovascular disease.

Free DNA levels have been considered to be a telling prognostic for these and other diseases, yet the methodology to quantitatively assess free circulating DNA levels is expensive and time consuming.

There is no effective biomarker for screening women for breast cancer, certainly there is no minimally invasive and reliable screen for breast cancer. The drawbacks to routine screening with mammography necessitate the identification of new decisive biomarkers.

### SUMMARY OF THE INVENTION

In one embodiment, a method of assessing breast cancer risk can comprise quantifying a DNA concentration in a blood sample from a subject. The method can further comprise mixing a blood sample with a fluorescent nucleic acid intercalating agent, wherein said mixing is conducted in the absence of prior nucleic acid extraction; detecting fluorescence of said fluorescent nucleic acid intercalating agent; and correlating detection of said fluorescence with a value, wherein if said value represents an increased concentration of nucleic acid in said biological fluid sample versus that of a standard sample then said subject has an elevated risk for breast cancer.

In one embodiment, the method further can comprise correlating results in said value with a predictive value indicative of cancer stage in said subject.

In some embodiments, if the value obtained exceeds 850 ng/ml, then said method can further comprise providing a recommendation for mammography of said subject.

In some embodiments, if the value obtained exceeds 1400 ng/ml, then said method can further comprise providing a recommendation to avoid removal of proximal lymph nodes in said subject.

In one embodiment, a method of assessing breast cancer severity in a subject can comprise quantifying the DNA concentration in a blood sample from a subject. The method can comprise mixing a blood sample with a fluorescent nucleic acid intercalating agent, wherein said mixing is conducted in the absence of prior nucleic acid extraction; detecting fluorescence of said fluorescent nucleic acid intercalating agent; and correlating detection of said fluorescence with a value, wherein if said value represents an increased concentration of nucleic acid in said biological fluid sample versus that of a standard sample then said subject has breast cancer of an advanced stage.

In some embodiments, a method of assessing responsiveness to a breast cancer treatment modality in a subject can comprise quantifying the DNA concentration in a blood sample from a subject by mixing a blood sample with a fluorescent nucleic acid intercalating agent, wherein said mixing is conducted in the absence of prior nucleic acid extraction; detecting fluorescence of said fluorescent nucleic acid intercalating agent; and correlating detection of said fluorescence with a value, wherein if said value represents an increased concentration of nucleic acid in said biological fluid sample versus that of a standard sample then a responsiveness of said subject to a breast cancer treatment modality is poor

In some embodiments, correlating detection of said fluorescence can be with a value representing a baseline determination taken from said subject prior to commencement of said treatment modality

In some embodiments, correlating detection of said fluorescence can be against a value obtained for a standard sample and said baseline determination,

In some embodiments, detecting in accordance with the methods as herein described can be conducted with the use of a fluorimeter. In one embodiment, the fluorescent nucleic acid intercalating agent comprises SYBR Gold® or SYBR Green®.

In one embodiment, the subject can have or can be predisposed to breast cancer. In one embodiment, the method can further comprise diagnosing the presence of breast cancer based on the value obtained. In one embodiment, the method can further comprise predicting the severity of breast cancer based on said value obtained. In one embodiment, the method can further comprise assessing response of a subject to treatment of breast cancer, based on said value obtained.

In one embodiment, a kit for assessing breast cancer risk in a subject can comprise: a fluorescent nucleic acid intercalating agent; a diluent; at least one positive standard sample comprised of a known concentration of DNA in said diluent; and optionally at least one negative standard sample comprised of a known concentration of DNA in said diluent; wherein said kit provides instructions for quantifying the relative fluorescence of a blood sample of a subject versus that obtained for said positive standard and correlating the same with a risk factor for said subject.

In one embodiment, the kit optionally comprises a container suitable for accommodating said series of solutions and said blood sample and wherein said container may be applied to a fluorimeter. In one embodiment, the fluorescent nucleic acid intercalating agent comprises SYBR Gold® or SYBR Green®.

All publications, patents, and patent applications mentioned herein are hereby incorporated by reference in their entirety as if each individual publication or patent was specifically and individually indicated to be incorporated by reference. In case of a conflict between the specification and an incorporated reference, the specification shall control. Where number ranges are given in this document, endpoints are included within the range. Furthermore, it is to be understood that unless otherwise indicated or otherwise evident from the context and understanding of one of ordinary skill in the art, values that are expressed as ranges can assume any specific value or subrange within the stated ranges, optionally including or excluding either or both endpoints, in different embodiments of the invention, to the tenth of the unit of the lower limit of the range, unless the context clearly dictates otherwise. Where a percentage is recited in reference to a value that intrinsically has units that are whole numbers, any resulting fraction may be rounded to the nearest whole number.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A-1D describe fluorescence as a function of DNA concentration, in serum samples probed with SYBR Gold. Concentrations of as little as 50 ng/ml of DNA were detected. Commercial Salmon sperm DNA was dissolved at various concentrations in four different fluids: FIG. 1A. 20% solution of DNase-treated pooled serum from 10 healthy donors in PBS. FIG. 1B. 2% solution of bovine serum albumin (BSA) in PBS. FIG. 1C. Fresh heparinized whole blood from a healthy donor and FIG. 1D. Pooled urine from 10 healthy donors. Urine was buffered to pH 7.4 with 10 mM HEPES. DNA solutions were added in duplicates to black 96 well plates, SYBR® Gold was added to each well (1:10000) and fluorescence was measured at 535 nM (F535) by a plate reader fluorometer.

FIG. 2A and FIG. 2B are side-by-side comparisons of fluorescence of SYBR Gold and SYBR Green mixed with serially diluted salmon DNA in 20% normal pooled human sera. FIG. 2C demonstrates DNA detection in whole blood with SYBR gold. FIG. 2D shows linear fluorescence intensity as a function of DNA concentration in the presence of the florescent dye EvaGreen®. For this experiments DNA was diluted in phosphate buffered saline (PBS) containing 2% bovine serum albumin (BSA) and EvaGreen®, was added in final dilution of 1:1000.

FIG. 3A describes fluorescence as a function of DNA concentration, in peritoneal lavage fluid collected from mice challenged intra-peritoneally with E. coli. FIGS. 3B-1 and 3B-2 demonstrate that DNA concentrations correlated well with the levels of IL-6 or TNF (FIGS. 3C-1 and 3C-2) markers that reflect the intensity of a destructive inflammatory process in lavage fluid and in serum.

FIG. 4A describes serum troponin correlation with DNA detection. FIG. 4B demonstrates that treatment of the serum sample with DNase abolished fluorescence. Random serum samples (0.5 ml) were treated with DNase (500 U) or RNase (100 U) (FIGS. 4C and 4D). FIG. 4C. Fluorescence of one representative serum. FIG. 4D. Fluorescence of sera after incubation with RNase (=5) or DNase (n=9) in relation to the corresponding sera not incubated with nuclease. *** indicates p<0.001. FIGS. 4E-G show DNA quantification of samples from hospitalized patients with acute myocardial infarction (MI) at different hours following their arrival at the emergency room of the hospital. FIG. 4H-J depict the DNA level (FIG. 4H), distribution, (FIG. 4I) and patient outcome (FIG. 4J) of 200 subjects who were evaluated in this setting, as compared to healthy volunteers.

FIGS. 5A-5D describe a side-by-side DNA quantification of samples in which DNA was subjected to a prior extraction step, or not. FIGS. 5A and 5B quantify DNA isolated from whole blood of a normal healthy donor and extracted, per the QIAamp DNA blood Kit (Qiagen) and quantified by SYBR Gold assay (FIG. 5A) or QPCR of the β-globin gene. FIG. 5C describes DNA quantification by SYBR Gold assay in serum, where the DNA was not subjected to a prior extraction step. FIG. 5D describes the linear correlation between the SYBR® Gold assay and β-globin QPCR assay. Human DNA was purified from leukocytes of a healthy volunteer and quantified by optical density (260 nM) using a nanodrop spectrophotometer. Serial dilution of DNA concentration was then determined by the SYBR® Gold assay (F535) and by real time PCR (QPCR) using specific primers for β-globin.

FIGS. 6A and 6B describe the effect of serum concentration on F535 background and quenching. Pooled human serum was preincubated with DNase and diluted with PBS to various concentrations; same amount of salmon sperm DNA was added to all solutions resulting in a final concentration of 1140 ng/ml. Serum solutions at same concentrations not containing DNA were used to determine background fluorescence. FIG. 6A. Total and background fluorescence of serum solutions FIG. 6B. Calculated % quenching of the specific DNA signal [100−100×(Total F535−Background F535)/total F535]. Assay was performed in triplicates, ** indicates p<0.01 comparing serum solution to PBS without serum.

FIGS. 7A and 7B demonstrate the effect of storage conditions on the assay. FIG. 7A. Blood from 7 healthy volunteers was collected into commercial gel tubes (8 tubes per donor). From each donor 5 tubes were stored at room temperature (RT) and 3 tubes at 4° C. Tubes were centrifuged and sera were collected for the DNA assay at indicated time points. FIG. 7B. Aliquots of 10 different sera (3 low, 4 elevated and 3 high DNA concentrations) were and incubated for 24 hrs at RT or frozen and thawed 5 times and then assayed for DNA. Assays were performed in triplicates. Readings of different time points were compared with readings at time zero. *** indicates p<0.001.

FIGS. 8A-8D show intra-day and intra-assay variation: To assess the variation of the assay, three sera were used containing normal, elevated and high DNA concentrations (197, 1096 and 4107 ng/ml, respectively) FIG. 8A. Intra-day variation of the assay was assessed by comparing readings of 12 assays of each sample in duplicates done independently on separate plates at different times over one working day. FIG. 8B. Day to day variation was assessed by comparing readings of 12 aliquots of each sample. Aliquots have been frozen and assayed on different days. For this assay, serum of three donors was used with low, elevated and high DNA concentrations (383, 1152 and 2735 ng/ml, respectively). Median value of the assays is indicated by the line inside the box. The Box indicates the distribution of 50% of the results and the bar above and below the box indicates 25% of the data. FIG. 8C Normal reference values were obtained by analysis of sera from 47 healthy volunteers. The volunteers were mostly students which declared to be healthy and with no chronic disease. The cohort consisted of 22 women and 25 man with an average age of 26.3±4.7 years. FIG. 8D. presents statistical analysis.

FIG. 9A demonstrates DNA quantification in subjects with viral infection, where DNA levels detected are higher in active EBV and CMV infection, as opposed to controls. FIGS. 9B-1-9B-4 demonstrate that DNA quantification correlated well with viral load in HIV infected patients. FIG. 9C demonstrates quantification in subjects with sepsis. FIGS. 9D and 9E demonstrate quantification in subjects with active peritonitis, and the correlation between leukocyte number in peritoneal fluid and DNA concentration.

FIG. 10 demonstrates quantification in a subject recovering from acute graft rejection following kidney transplantation, with DNA levels correlating well with creatinine levels.

FIGS. 11A-11I demonstrate quantification of circulating DNA levels in trauma patients and its correlation with clinical complications arising in particular subjects.

FIGS. 12A and 12B show DNA quantification in cancer subjects and cancer models. FIG. 12A. CFD levels were elevated in patients with colon cancer, one week before tumor removal. FIG. 12B. Elevated circulating DNA levels correlate with tumor size in mice inoculated intra footpad with an MCA-2 fibrosarcoma cell line with 1.0×106 cells/mouse (N=10).

FIGS. 13A-13C show assay efficacy on cell lysates. Cultured fibroblast cells (L-cells) seeded at various numbers in triplicates (0, 40, 60, 80, 100, 120, 150 and 200×103 cells/well) in 24 well plates with 1 ml of DMEM medium containing 10% fetal calf serum. Cell lysis was induced with a detergent (0.1% NP40) and gentle agitation for 30 minutes. Supernatants were collected and assayed for free DNA and LDH activity. FIG. 13A. Supernatant free DNA (F535). FIG. 13B. Supernatant LDH activity. FIG. 13C. Correlation between supernatant free DNA and LDH activity.

FIGS. 14A and 14B show CFD levels in patient with breast cancer. FIG. 14A. CFD levels were measured in 34 unselected patients with confirmed primary early stage breast cancer before surgery and 9 patients 1 month after surgery. CFD reference values were established by analysis of sera from 18 healthy female volunteers. Horizontal lines indicate median of each group. Dashed line indicates the CFD cutoff value (850 ng/ml). *** indicates P<0.001, ** P<0.01. FIG. 14B. Receiver operating characteristic (ROC) analysis of breast cancer patient compared to control group. Calculated area under the curve (AUC)=0.81 (95% CI, 0.69 to 0.93).

FIG. 15 provides a comparison between pre-surgery-CFD and -CA15.3 levels in patients with early stage breast cancer. CFD and CA15.3 levels were measured in 34 unselected patients with confirmed primary early stage breast cancer before surgery.

FIGS. 16A and 16B show the CFD distribution as a function of the presence of receptors.

FIG. 17 shows the CFD distribution according to tumor size. Empty symbols indicate patients with auxiliary nodes involvement. * indicates P<0.05.

FIGS. 18A-18C show CFD levels as a function of lymph node involvement. FIG. 18A. CFD distribution according to grading of lymph node involvement, (0) no involvement, (1) 1-4 lymph nodes (2) >4 lymph nodes. FIG. 18B. Comparisons between CFD levels of tumor with (N+) or without node involvement (N−). FIG. 18C. ROC analysis of CFD levels in breast cancer patient with node involvement compared to control group. Calculated AUC of 0.97 (95% CI, 0.93 to 1.02). * P<0.05, **P<0.01, ***p<0.001.

FIG. 19 shows CFD distribution according to tumor stage.

### DETAILED DESCRIPTION OF THE PRESENT INVENTION

This invention provides methods and materials for the assessment of breast cancer risk, assessing breast cancer severity in a subject and/or assessing responsiveness to a breast cancer treatment modality in a subject.

In some embodiments, the methods detect and quantify free DNA in blood samples, and in some embodiments, in other biological samples. In some embodiments, the term ‘free DNA’ refers to extracellular deoxynucleic acids, for example unbound DNA or circulating nucleic acids as present in bodily fluids as defined above. The DNA can, nevertheless, be bound to proteins in the bodily fluid, this will also be understood to represent embodiments of “free DNA” in the context of the present invention. In some embodiments, the DNA free in the bodily fluid is derived from single cells or clumps of cells that are derived from organs or tissues (e.g. lung cells that are expectorated) and have lysed, releasing their DNA. The DNA that is released from these cells in said bodily fluid will also be understood as “free DNA” in the context of the present invention.

The invention provides methods and/or kits for quantifying free DNA in bodily fluids. In some embodiments, the term “biological fluid” refers to a liquid taken from a biological source and includes, for example, blood, serum, plasma, sputum, lavage fluid, cerebrospinal fluid, urine, semen, sweat, tears, saliva, or others.

In some embodiments, the bodily fluid refers to whole blood, blood plasma, blood serum, urine, sputum, ejaculate, semen, tears, sweat, saliva, lymph fluid, bronchial lavage, leukophoresis samples, pleural effusion, peritoneal fluid, meningal fluid, amniotic fluid, glandular fluid, fine needle aspirates, nipple aspirate fluid, spinal fluid, conjunctival fluid, vaginal fluid, duodenal juice, pancreatic juice, bile, cerebrospinal fluid or mucus secretions from a desired subject. In some embodiments, the term “fluid” for sample in the methods and via the kits of this invention, refers to a tissue homogenate, cell culture or bioreactor fluid sample, as described further hereinbelow.

In some embodiments, the biological fluids are from mammalian subjects, where a sample may be obtained from differing sources, including, but not limited to, samples from different individuals, different developmental stages of the same or different individuals, different diseased individuals (e.g., individuals with cancer or suspected of having a genetic predisposition to cancer), normal individuals, different disease stages of the same or different individuals, individuals subjected to different disease treatment, individuals subjected to different environmental factors, or individuals with predisposition to a pathology.

In some embodiments, it is desirable to recover the DNA from bodily fluids, tissues or excretions located proximally to the disease site. Such samples may be derived, for example, from feces, urine, blood, sputum, biopsy or resection sites, or other sources.

In one embodiment, the sample is collected from a pregnant female, for example a pregnant woman. According to this aspect, and in one embodiment, the sample can be analyzed using the methods described herein to prenatally diagnose chromosomal abnormalities in the fetus. The sample can be collected from biological fluids, for example the blood, serum, villus sampling, or some fraction thereof.

In some embodiments, reference to the terms “blood.” “plasma” and “serum” are to be taken to expressly encompass fractions or processed portions thereof. Similarly, where a sample is taken from a biopsy, swab, smear, etc., the “sample” expressly encompasses a processed fraction or portion derived from the biopsy, swab, smear, etc.

In some embodiments, the bodily fluid is obtained from a single subject or individual. In some embodiments, the term “individual” or “subject” refers to a human subject as well as a non-human subject such as a mammal, an invertebrate, a vertebrate, a rat, a horse, a dog, a cat, a cow, a chicken, a bird, a mouse, a rodent, a primate, a fish, a frog, a deer. The examples herein are not meant to limit the methodology of the present invention to a human subject only, as the instant methodology is also useful in the fields of veterinary medicine, animal sciences, research laboratories and such.

In some embodiments, the method comprises mixing a blood or other biological fluid sample with a fluroescen nucleic acid intercalating agent.

In some embodiments, the term “mixing” refers to contact proximity, for example, dispensing of a fluid detectable nucleic acid intercalating agent in a container containing a sample of the bodily fluid, or vice versa. In some embodiments, mixing may comprise more extensive agitation of the fluid, with any aid, such as, for example, conventional mixers, the use of stirring aids, the use of vortex machinery, sonication, or any means known in the art. No means of mixing is to be considered precluded, nor is any limitation imposed upon the time or amount of mixing necessary for the creation of proximity between the fluid sample and the intercalating agent, such that the intercalating agent may intercalate within nucleic acids present in the bodily fluid sample.

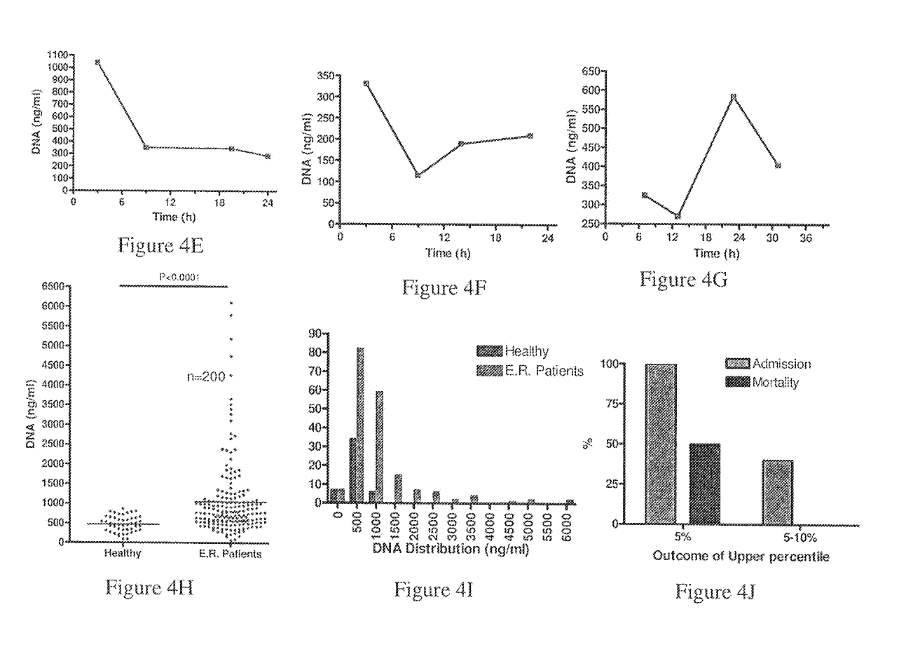

Surprisingly, in the present invention, it was found that contacting biological fluid samples comprising nucleic acids, with a nucleic acid intercalating agent, without prior nucleic acid extraction was as sensitive, and in some embodiments, more sensitive, in quantifying the nucleic acid concentration in the sample, than samples whose nucleic acids had been previously subjected to extraction (FIG. 5). The lack of necessity for such an extraction step increases efficiency and ease of the quantification assay, and reduces cost, such that there is a clear advantage to such assays.

Furthermore, most surprisingly, the methods as described herein when applied to the detection of early-stage breast cancer demonstrated a 51.5% with a 95% CI and a specificity of 100%. The methods demonstrated that the results provide a strong correlation with tumor size, nodal involvement and cancer stage, with a sensitivity for detection of cancer patients with nodal involvement (N+) of 82% and a sensitivity of detection for Stage 3A of 100%.

The methods and kits of this invention make use of a nucleic acid intercalating agent. In some embodiments, the term “intercalating” or grammatical forms thereof refers to the insertion of a compound between adjacent base pairs of a strand of DNA. For example, and in some embodiments, the term “intercalating” refers to the insertion of planar aromatic or heteroaromatic compounds between adjacent base pairs of double stranded DNA (dsDNA).

In some embodiments, the intercalating agent is one in which a change in fluorescence occurs upon binding to a nucleic acid. In some embodiments, the intercalating agent is one which fluoresces upon binding to DNA, or in some embodiments exhibits a marked increase in fluorescence upon DNA binding.

In some embodiments, the intercalating agent is a phenanthridium compound, as described in U.S. Pat. Nos. 5,436,134, 5,582,984, 5,808,077, 5,658,751, 6,664,047, fully incorporated herein in their entirety. In some embodiments, the intercalating agent is a cyanine compound, for example a dimeric cyanine stain. In some embodiments, the cyanine compound is a SYBR® stain, Picogreen®, Oligreen® or Ribogreen® or a POPO®, BOBO®, YOYO®, TOTO®, JOJO® or LOLO® stains or TO-PRO® stains (Molecular Probes/Invitrogen Inc.) or EvaGreen®. In some embodiments, the intercalating agent is ethidium bromide, propidium iodide, Quinolinium, 1-1′-[1,3-propanediylbis[(dimethyliminio)-3,1-propanediyl]]bis[4-[(3-methyl-2(3H)-benzothiazolylidene)methyl]]-,tetraiodide}, or 4-[(3-methyl-2(3H)-benzoxazolylidene)methyl]-1-[3-(trimethylammonio) propyl]-,diiodide}.

The intercalating agents used in the methods/kits of this invention comprise a detectable moiety. In some embodiments, the phrase “comprise a detectable moiety” refers to the association of the moiety with the intercalating agent, for example by covalent or non-covalent bonds. In some embodiments, the phrase “comprise a detectable moiety” refers to the agent itself being detectable, for example, the agent itself fluoresces upon DNA binding.

The methods comprise detecting the moiety by any means known in the art, suitable for detection of the particular moiety. For example, and in some embodiments, the detectable moiety is fluorescent upon binding, and detection is accomplished with the aid of a fluorimeter. In some embodiments, spectrophotometric detection may be used for detectable changes in absorbance upon interaction of the detectable moiety with the nucleic acid. In some embodiments, detection is by any means, for example, automated means, wherein changes in physical properties of the sample are quantifiable.

The methods of this invention comprise correlating the detected value with one reflective of the concentration of nucleic acid in the bodily fluid sample. In some embodiments, the detected changes are quantified and correlated with the concentration of nucleic acids, for example as described herein in Examples 1-4. In some embodiments, the detectable moiety is fluorescent, and fluorescence is measured quantitatively in a fluorimeter, and the values obtained for a particular sample are compared to a series of standards, whose nucleic acid concentration is known. According to this aspect and in one embodiment, fluorescence of the standards is determined, under identical conditions as those applied for the sample. According to this aspect and in one embodiment, the sample fluorescence is determined, and the nucleic acid concentration is derived, based on comparability of the fluorescence for known nucleic acid concentrations of the series of standards tested. In some embodiments, a standard curve is derived for the nucleic acid concentration of the standards plotted as a function of fluorescence obtained, and thereby sample concentrations can be determined.

In one embodiment, the method is conducted in parallel to mixing a second bodily fluid sample obtained from a second subject, and said correlating results in said value representing a standard for said method. According to this aspect, and in one embodiment, a first sample nucleic acid is being determined relative to a second sample, whose concentration is not necessarily known, but the status of the source for the nucleic acid material from the second sample serves as a negative standard for the first sample, such that quantification, for example, fluorescence levels, if significantly lower than those obtained from the second sample serve as a diagnostic or prognostic indicator for the subject from whom the first sample was obtained, for example, no disease, or for example, desirable response to therapy, and others as will be appreciated by one skilled in the art.

Similarly, in some embodiments, if the quantification, for example, fluorescence levels, yields values significantly higher than those obtained from the second sample serve as a diagnostic or prognostic indicator for the subject from whom the first sample was obtained, for example, presence of disease, or for example, exacerbation of disease, or for example, poor response to therapy, and others as will be appreciated by one skilled in the art. In some embodiments, a series of standards is generated representing severity of a disease or condition, such that increasing values obtained for fluorescence represents discrete stages in disease pathogenesis, for example cancer staging values, and evaluation of a particular sample under identical conditions, in comparison to the series of standards serves as a diagnostic both for the presence and staging of a cancer, according to this aspect.

It will be appreciated that various standards may be employed, whereby the obtained results from a particular sample, when compared to those obtained for the series of standards will serve as a diagnostic or prognostic indicator, as a function of the quantitative result obtained, and/or as a function of a relative value to those obtained in the series of standards.

In one embodiment, the subject has or is predisposed to a disease or disorder. According to this aspect, and in one embodiment, the subject has a genetic predisposition to a cancer, or in another embodiment, the subject has certain lifestyle risk factors associated with a cancer or other related disease, or in another embodiment, the subject exhibits phenotypic characteristics or symptoms associated with incidence of cancer or a related disease.

In one embodiment, the method further comprises diagnosing the presence of said cancer or a related disease based on said value obtained.

In some embodiments, if the value obtained exceeds 850 ng/ml, then said method further comprises providing a recommendation for mammography of said subject.

In some embodiments, if the value obtained exceeds 1400 ng/ml, then said method further comprises providing a recommendation to avoid removal of proximal lymph nodes in said subject.

According to these aspects, and in some embodiments, pre-screening subjects in accordance to the methods of this invention provides a means for screening prior to providing a recommendation for performing mammography, which is a more focused screen than one based on age, for example, with common clinical practice recommending performing mammography in all women over 50 years of age every two years. Other advantages of the embodied pre-screening method is the ability to reduce the incidence of over diagnosis, to reduce overall population screening costs and reduce discomfort for the screened patients.

According to these aspects, and in some embodiments, pre-screening subjects in accordance to the methods of this invention and identifying subjects with an elevated CFD over a cutoff, which in some embodiments, is over 1400 ng/ml, or in some embodiments, over 1200 ng/ml, or in some embodiments, over 1150 ng/ml, or in some embodiments, over 1100 ng/ml, or in some embodiments, over 1060 ng/ml, or in some embodiments, over 1030 ng/ml, or in some embodiments, over 1000 ng/ml, or in some embodiments, over 975 ng/ml, provides a method which will replace lymph node removal used as a supplemental corroboratory screen to date, a surgery that cause a lot of discomfort and significant morbidity.

In one embodiment, the method further comprises predicting the severity of said cancer or a related disease based on said value obtained.

In one embodiment, the method further comprises assessing response of a subject to treatment of said cancer or a related disease, based on said value obtained.

In one embodiment, the disease or disorder comprises neoplasia or preneoplasia.

In some embodiments, this invention provides a method to determine the presence or absence of a medical condition such as cancer. The method employs, inter alia retrieval of an individual's sample in form of a biological fluid like blood, serum, urine or other fluids as described herein, and others known in the art. In some embodiments, the method employs determining the amount of free DNA in the sample, with the amount or presence (detectable above a given threshold) of free DNA serving as a diagnostic or prognostic indicator, i.e. from this determination, in some embodiments, the presence or absence or severity of a medical condition can be concluded.

In some embodiments, the methods/kits of this invention enable the prediction of whether an individual suffers from, or is at risk for a particular medical condition, such as cancer or a related disease. In some embodiments, once alterations in nucleic acid levels are rapidly detected, the nucleic acid is further probed for additional characteristics, which in turn may further elucidate for example, not just the presence of a proliferative disease, but the source, e.g. tissue of the proliferative cells. Such secondary determinations may be conducted by any means known in the art, for example by PCR technology, with probes specific for detecting certain characteristic genes, for example, or for example for detecting certain methylation patterns, or others, as will be appreciated by one skilled in the art.

The methods of this invention, in some embodiments, involve a biological fluid sample being retrieved from a patient or individual. The retrieval of the said sample may be conducted via any means known to a person skilled in the art. In some embodiments, such retrieval may comprise, inter alia, ventricular puncture, also known as CSF collection, a procedure to obtain a specimen of cerebrospinal fluid (CSF); thoracentesis, referring to inserting a needle between the ribs into the chest cavity, using a local anesthetic to obtain the pleural effusion fluid; amniocentesis, referring to a procedure performed by inserting a hollow needle through the abdominal wall into the uterus and withdrawing a small amount of fluid from the sac surrounding the fetus, or standard means for blood, urine, sperm or sputum collection, or other means.

In some embodiments, nucleic acid quantification as described herein may take place either immediately after retrieval of the sample or after an unspecified time of storage of said sample.

In some embodiments, the methods/kits of this invention find use in the identification of subjects with abnormal amounts of free nucleic acid, with normality being a function of the absence of a deviance of 10% or more from a value defined as “normal”, in their bodily fluids. In some embodiments, normality is a function of the absence of a deviance of 20% or more from a value defined as “normal”, in their bodily fluids, or in some embodiments, normality is a function of the absence of a deviance of 30% or more from a value defined as “normal”, in their bodily fluids, or in some embodiments, normality is a function of the absence of a deviance of 40% or more from a value defined as “normal”, in their bodily fluids.

In some embodiments, such deviance serves as a diagnostic or prognostic indicator. In some embodiments, the term “diagnostic” and grammatical forms thereof, when referred to herein, refers to the ability to demonstrate an increased likelihood that an individual has a specific condition or conditions. In some embodiments, diagnosis also refers to the ability to demonstrate an increased likelihood that an individual does not have a specific condition. In some embodiments, diagnosis refers to the ability to demonstrate an increased likelihood that an individual has one condition as compared to a second condition. In some embodiments, diagnosis refers to a process whereby there is an increased likelihood that an individual is properly characterized as having a condition (“true positive”) or is properly characterized as not having a condition (“true negative”) while minimizing the likelihood that the individual is improperly characterized with said condition (“false positive”) or improperly characterized as not being afflicted with said condition (“false negative”).

In some embodiments, the term “prognostic” and grammatical forms thereof, when referred to herein, refers to the ability to predict the progression or severity of a disease or condition in an individual. In some embodiments, prognosis also refers to the ability to demonstrate a positive response to therapy or other treatment regimens, for the disease or condition in the subject. In some embodiments, prognosis refers to the ability to predict the presence or diminishment of disease/condition associated symptoms.

In some embodiments, the methods/kits described herein find application in diagnostic and prognostic purposes, wherever it is advantageous to determine the relative or absolute amount of nucleic acid in a sample.

In some embodiments, the methods/kits described herein find application in the diagnosis and/or prognosis of cell proliferative diseases in a subject. In some embodiments, such proliferative diseases comprise cancers, including but not limited to biliary tract cancer, brain cancer, including glioblastomas and medelloblastomes; breast cancer, cervical cancer; choriocarcinoma; colon cancer, endometrial cancer, esophogeal cancer, gastric cancer, hematological neoplasms, including acute lymphocytic and myelogeneous leukemia, multiple myeloma, AIDS associates leukemias and adult T-cell leukemia lymphoma; intraepithelial neoplasms, including Bowen's disease and Paget's disease; liver cancer; prostate cancer, lung cancer; lymphomas, including Hodgkin's disease and lymphozytic lymphomas; neuroblastomas; oral cancer, including squamous cell carcinoma; ovarian cancer, including those arising from epithelial cells, stromal cells, germ cells and mesenchymal cells; pancreas cancer; rectal cancer, ovarian cancer, sarcomas, including leiomyosarcoma, rhabdomyosarcoma, liposarcoma, fibrosarcoma and osteosarcoma; skin cancer, including melanoma, Kaposi's sarcoma, basocellular cancer and squamous cell cancer; testicular cancer, including germinal tumors (seminoma, non-seminoma (teratomas, choriocarcinomas)), stromal tumors and germ cell tumors; thyroid cancer, including thyroid adenocarcinoma and medullar carcinoma; renal cancer including adenocarcinoma and Wilms tumor and others.

In one embodiment, this invention provides a kit for assessing breast cancer risk in a subject, said kit comprising: a fluorescent nucleic acid intercalating agent; a diluent; at least one positive standard sample comprised of a known concentration of DNA in said diluent; and optionally at least one negative standard sample comprised of a known concentration of DNA in said diluent; wherein said kit provides instructions for quantifying the relative fluorescence of a blood sample of a subject versus that obtained for said positive standard and correlating the same with a risk factor for said subject.

It is to be understood that the intercalating agents, series of solutions comprising standards, etc. may comprise any embodiment thereof as described herein, and any others appropriate, as will be appreciated by the skilled artisan.

The diluent described herein may be any suitable solvent or solution, which serves to dilute the sample as desired, and wherein the properties of the diluent do not interfere with the detection and/or quantification of the nucleic acid in the sample. In some embodiments, the diluent is any suitable buffer, or solution, for example, physiological saline, or for example, phosphate buffered saline, and others as will be appreciated by the skilled artisan.

In one embodiment, the kit optionally comprises a container suitable for accommodating the series of solutions and said bodily fluid sample and wherein the container may be applied to a fluorimeter. In some embodiments, the methods/kits of this invention lend themselves to automation, and standard assay dishes and plates, for example 96 well plates commonly sold by commercial vendors are suitable for use. In some embodiments, the apparatus utilized for the detection as described herein, will accommodate such containers readily, further adding to the ease and cost-effectiveness of the kits/methods described herein.

The kits may be formatted for use in a diagnostic apparatus (e.g., an automated analyzer) or can be self-contained (e.g., for a point-of-care diagnostic).

In one embodiment, the kit comprises a container suitable for the assay of urine, blood or a component thereof, lavage fluid or a combination thereof. Biological fluids often represent a hazard for the technician assaying the same, and various means have been developed to minimize exposure and thereby risk to the technician performing the assay, for example transfer with plastic, non-sharp transfer means of the fluid to the assay container, seals for such containers, etc. In one embodiment of this invention, as the assay provides for rapid quantification, kits are particularly constructed such that as many safety precautions as possible are employed for use with the sample fluids to minimize risk while maximizing speed in effecting the methods of this invention.

Kits for determining the quantities of nucleic acids will comprise one or more containers holding reagents useful for performing the assays, including, for example, containers holding standards and intercalating agents. Suitable containers for the reagents of the kit include bottles, vials, test tubes and microtiter plates. Also, reagents (e.g., intercalating) can be incorporated into or onto substrates, test strips (made of, e.g., filter paper, glass, metal, plastics or gels) and other devices suitable for performing the assay. Instructions for performing one or more assays for quantifying nucleic acid will be provided with the kits (e.g., the instructions can be provided in the same package holding some or all of the reagents or can be provided in separate documentation). The kit may also contain other materials which are known in the art and which may be desirable from a commercial and user standpoint, such as buffers, enzyme substrates, diluents, standards, etc. Finally, the kit may include containers, such as empty containers for performing the assay, for collecting, diluting and/or measuring a body fluid, and/or for diluting reagents, etc.

Kits for diagnosing diseases or conditions described herein will comprise one or more containers holding reagents useful for the same, including secondary assay materials/reagents for further identification, once the initial finding of altered nucleic acid concentration is ascertained. Such kits may be two-part kits, each part providing the reagents and other materials for performing one of the assays. Instructions for performing each of assays will be provided with the kits (e.g., the instructions can be provided in the same package holding a two-part kit, can be provided in each of the packages holding the separate kits, or can be provided in separate documentation).

The kit may comprise a container holding a color-producing material (i.e., a material capable of undergoing a color-producing reaction when contacted with the intercalating agent). Such a kit may further comprise a container for collection of a body fluid (such as a syringe or a plastic or paper cup), an instrument for measuring the body fluid (such as a dropper, a pipette or a micropipette) and either a color comparison chart or containers holding standards comprising known amounts of nucleic acid.

Current breast cancer screening methods rely on breast self-examination (BSE), clinical breast examination (CBE) and mammography, with mammography being considered to the most important tool in the early diagnosis of breast cancer. Nonetheless, while being a highly sensitive and specific diagnostic tool, considerable debate as to its reliability exists in the medical community with a hypothesized screened population deriving a risk reduction of 0.05% in mortality breast cancer while concurrently increasing mortality by 0.5% due to over diagnosis and overtreatment.

Surprisingly, it was found that a simple and straightforward method to measure CFD levels provided a highly sensitive and accurate means of detecting cancer and even identifying a likelihood of nodal involvement and an indication of cancer stage, aspects that heretofore have not been possible in a non-invasive, rapid assay.

Comparisons between the CFD levels in breast cancer patients to that of CA15.3 positivity demonstrated a much higher specificity and sensitivity (100% and 52%, respectively), whereas CA15.3 8.6%. There has been no foundation for the increased potential reliability for circulating DNA levels providing greater sensitivity than serological CA15.3 positivity.

More surprisingly, CFD levels correlated with tumor size and provided a highly sensitive and reliable assay for detection lymph nodes involvement (ROC AUC=0.97).

It is known that of all risk factors, the number of axillary nodes involved in breast cancer provides the highest correlation in terms of risk for distant metastasis recurrence in women with primary nonmetastatic breast cancer. That circulating DNA levels can be used as a prognostic indicator for nodal involvement and metastatic potential is unprecedented and contraintuitive and represents a surprising result of the instant invention.

In some cases, it is known that small tumors at the primary site (TI, <2 cm) may still in axillary nodal involvement. To date, such small tumors represented a diagnosis challenge since they tend to be missed at palpation and may be of risk especially to non-mammography screened patients.

Nonetheless, the highly sensitive methods of this invention provided for three such cases of determined nodal involvement based on CFD findings, which would likely have gone undetected by other methods.

Tumor stage remains the most important determinant of the outcome for women with breast cancer. Tumor stage correlated positively with CFD levels and the percent of CFD positive samples gradually increased from 30% for patients at stage 1 to 100% for stage 3A. This gradual increase in sensitivity provides a framework for retesting initially negative patients with an undetectable early stage event to increase the likelihood of detection at a later stage, where the subject would still be identified within a timeframe to facilitate effective treatment and thereby survival.

Most surprising, post-surgery cohort patients were all CFD negative, providing a robust means for individual follow-up, and the ability to determine the efficacy of various treatment modalities.

It will be understood by those skilled in the art that various changes in form and details may be made therein without departing from the spirit and scope of the invention as set forth in the appended claims. Those skilled in the art will recognize, or be able to ascertain using no more than routine experimentation, many equivalents to the specific embodiments of the invention described herein. Such equivalents are intended to be encompassed in the scope of the claims.

In the claims articles such as “a.”. “an” and “the” mean one or more than one unless indicated to the contrary or otherwise evident from the context. Claims or descriptions that include “or” or “and/or” between members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides, in various embodiments, all variations, combinations, and permutations in which one or more limitations, elements, clauses, descriptive terms, etc., from one or more of the listed claims is introduced into another claim dependent on the same base claim unless otherwise indicated or unless it would be evident to one of ordinary skill in the art that a contradiction or inconsistency would arise. Where elements are presented as lists, e.g. in Markush group format or the like, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in haec verba herein. Certain claims are presented in dependent form for the sake of convenience, but Applicant reserves the right to rewrite any dependent claim in independent format to include the elements or limitations of the independent claim and any other claim(s) on which such claim depends, and such rewritten claim is to be considered equivalent in all respects to the dependent claim in whatever form it is in (either amended or unamended) prior to being rewritten in independent format.

### EXAMPLES

### Materials and Methods

CFD is detected with the present assay directly in biologic fluids. SYBR® Gold Nucleic Acid Gel Stain, (Invitrogen, Paisley, UK) was diluted first at 1:1000 in DMSO and then at 1:8 in phosphate buffered saline (PBS, Biological Industries, Beth Haemek, Israel). 10 μl of DNA solutions were applied to a black 96 wells plate (Greiner Bio-One, Frickenhausen, Germany). 40 μl of diluted SYBR® Gold was added to each well (final dilution 1:10,000) and fluorescence was measured with a 96 well fluorometer (Spectrafluor Plus, Tecan, Durham, N.C.) at an emission wavelength of 535 nm and an excitation wavelength of 485 nm. EvaGreen (PCR-352, Jena Bioscience, Jena Germany) was used to stain DNA standards (PBS+2% BSA diluted in 96 well plates) at a 1:1000 dilution and fluorescence was measured at 535, same conditions as SybrGold).

### Background Fluorescence of Serum

For assessment of background reading and to establish the optimal serum concentration in DNA standard solutions, we used pooled human serum from ten healthy donors. Sera were preincubated at 37° C. overnight with either RNase (100 U, Sigma-Aldrich) or for 5 hours with DNase (500 U, 5-PRIME, Gaithersburg, USA). DNase was inactivated by 20 mM EDTA prior to addition of DNA standards.

For assessment of background and quenching of serum, serum was diluted with PBS to various concentrations (0, 10, 20, 30 and 40% serum, respectively); same amount of salmon sperm DNA was added to all solutions resulting in a final DNA concentration of 1140 ng/ml. Assay was performed in triplicates. Serum solutions at same concentrations not containing DNA were used to determine background fluorescence.

### DNA Standards

For the fluorometric assay, standards were prepared with commercial Salmon sperm DNA (Sigma-Aldrich, Rehovot, Israel). For comparison with the conventional QPCR assay, human DNA was extracted from blood leukocytes using QIAamp Blood Kit (Qiagene, Hilden, Germany) according to the manufacturer's protocol. Concentrations of DNA used for the standard curves were determined by UV absorbance at 260 nm using a Nano-Drop spectrophotometer (Thermo Fisher Scientific, Wilmington, Del. USA).

### Biological Fluids

To assess the dose response of the assay in different fluids, salmon sperm DNA was diluted at various concentrations in four different fluids: A. 20% solution of pooled serum from 10 healthy donors in PBS. B. 2% solution of bovine serum albumin (BSA, Biological Industries, Beth Haemek, Israel) in PBS. C. Heparinized fresh whole blood from a healthy donor and D, pooled urine from 10 healthy donors buffered to pH 7.4 with 10 mM HEPES (Biological Industries).

### Effect of Storage Conditions

Whole Blood: Refrigeration vs. Room Temperature

To assess the effect of storage temperature on the assay, eight whole blood samples were collected from healthy volunteers into commercial gel tubes using the BD Vacutainer® system (Becton, Dickinson and Company, Plymouth, UK). Centrifugation was postponed; 5 tubes from each donor were stored for 0.5, 1. 4, 5 and 24 hours at room temperature (RT) and 3 tubes were stored for 0.5, 4, 24 hours at 4° C. At respective time points, tubes were centrifuged, sera were collected and assayed for DNA by the direct SYBR® Gold assay.

### Serum: Room Temperature

In a further experiment, aliquots of one human serum were incubated at RT and each aliquot was assayed at different time points. In addition, 10 random sera from our serum bank have been grouped according to their DNA level as measured by the direct SYBR® Gold assay into one of three groups: low, elevated and high range of DNA level. (low range group: 580, 460, 475 ng/ml; elevated range group: 2410, 2180, 2440, 2005 ng/ml; high range group: 3515, 3975, 3570 ng/ml). Sera have been thawed and aliquots were incubated for 24 hrs at RT and compared for their DNA levels with their corresponding aliquots which were kept at −20° C.

### Serum: Repeated Freezing and Thawing

Aliquots of the same 10 sera were frozen and thawed 5 times and compared for their DNA levels with their corresponding aliquots which were kept at −20° C. and thawed only once.

### Within-Day Variation

Intra-day variation of the assay was assessed by comparing readings of 12 assays done independently on separate plates at different times over one working day. In each assay, duplicates of 3 sera with low, elevated and high DNA concentrations (197, 1096 and 4107 ng/ml, respectively) were analyzed.

### Day to Day Variation

Day to day variation was assessed by comparing readings of aliquots from same sera on 4 different days. A total of 12 aliquots were analyzed in duplicates from three donors with low, elevated and high DNA concentrations (383, 1152 and 2735 ng/ml, respectively).

Comparison with Conventional CFD Assay

We also compared the direct SYBR® Gold assay with a conventional method of CFD assay as follows: Standards of human DNA were analyzed by quantitative real-time PCR (QPCR) amplification of the β-globin gene. The amplification mixture contained: 7 μl of DNA samples or human DNA standards (15-1000 ng/ml) in QIAamp elution buffer 2 μl of each primer (20 μM), 10 μl of ABsoluteBlue QPCR SYBR Mix Rox (ABgene, Surrey, UK) and water to a final volume of 20 μl. The primers and QPCR conditions of the human β-globin gene have been previously described (Jung M, Klotzek S, Lewandowski M, Fleischhacker M, Jung K. Changes in concentration of DNA in serum and plasma during storage of blood samples. Clin Chem 2003; 49:1028-9): Forward primer: 5′-ACACAACTGTGTTCACTAGC-3′ (SEQ ID NO: 1). Reverse primer: 5′-CAACTTCATCCACGTTCACC-3′(SEQ ID NO: 2).

The reaction was carried out in a Rotor-Gene real time PCR machine (Corbett-Research, Northlake, Australia). Cycle conditions were: initial activation step at 95° C. for 15 min, followed by 45 cycles of denaturation at 95° C. for 15 s, annealing at 56° C. for 20 s, and extension at 72° C. for 15 s. In parallel, the human standards were diluted in 20% DNase-treated pooled sera and assayed by the direct SYBR® gold assay. Correlation between the direct SYBR® Gold assay and the QPCR assay was assessed by the Spearman rank test.

### Reference Values

Reference value*s were established by analysis of sera from 47 healthy volunteers. The volunteers were mostly students which declared to be healthy and without chronic disease. The cohort consisted of 22 females and 25 males with an average age of 26.3±4.7 years. Three samples were excluded from the reference group: two of them because of hemolysis and one because the donor was diagnosed with acute infectious mononucleosis

Culture Media DNA after Cell Lysis

Cultured fibroblast cells (L-cells) were seeded at various numbers in triplicates (0, 40, 60, 80, 100, 120, 150 and 200×103 cells/well) in 24 well plates with 1 ml of DMEM medium containing 10% fetal calf serum (Biological Industries). Cell lysis was induced with a detergent (0.1% NP40) and gentle agitation for 30 minutes. Supernatants were collected and assayed for DNA concentration by the direct SYBR® Gold assay. In addition, LDH activity was assayed in the supernatant using a commercial kit (BioVision, Mountain View, Calif., USA) according to the manufacturer's protocol.

### Statistics

Statistic analysis was performed with GraphPad Prism® software (edition 4.01), Statistical significance was determined by t-test or analysis of variance. Significance of correlation was analyzed by Pearson-r test. A p-value<0.05 was considered significant.

Peritoneal lavage fluid and blood were collected from CD1 female mice aged 10 to 12 weeks (Harlan, Jerusalem, Israel) suffering from peritonitis 24 hours following induction by intraperitoneal E. coli inoculation with a sub-lethal dose (3.6×10⁹ CFU) as well as from age- and weight-matched controls. Serum was collected from human patients being hospitalized for a myocardial infarction or suspected thereto. Serum was collected from healthy donors, as well, serving as controls. Serum/lavage fluid was diluted 1:5 in PBS, and applied to 96 well tissue culture dishes.

Serially diluted (PBS) samples of known quantities of salmon DNA in 20% normal pooled human sera or whole blood were similarly applied to tissue culture dishes with 1:10000 dye.

SYBR Gold or SYBR green was added to each well such that the final dilution of the fluorochrome was 1:10,000. Fluorescence was assessed in a Fluorimeter, with excitation at 485 nm, emission at 535 nm.

### Example 1

### Rapid DNA Quantification in Biological Fluid

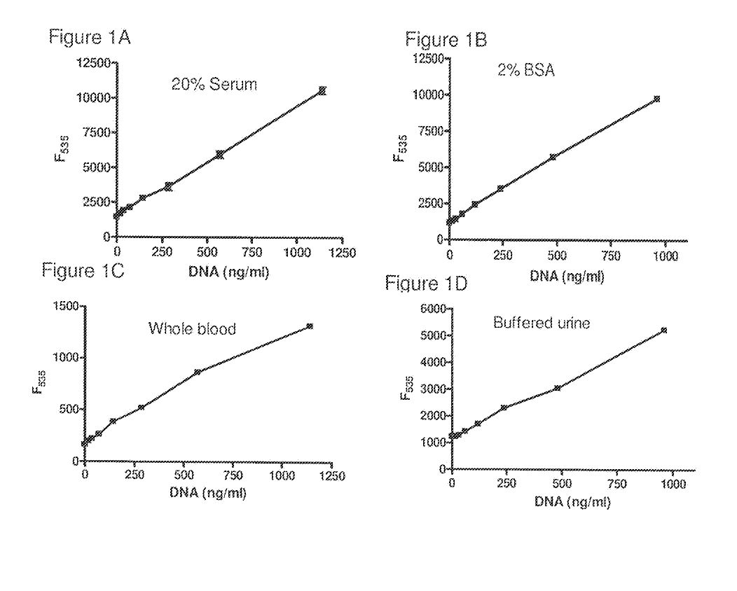

In order to determine whether rapid DNA quantification was obtainable using a DNA intercalating moiety, serum containing known dilutions of DNA was mixed with SYBR Gold, and fluorescence was determined (FIG. 1A). Concentrations of as little as 100 ng/ml of DNA were detected. Linearity was observed when 2% BSA, whole blood or buffered urine containing known dilutions of DNA were mixed with SYBR Gold as well (FIGS. 1B, 1C and 1D).

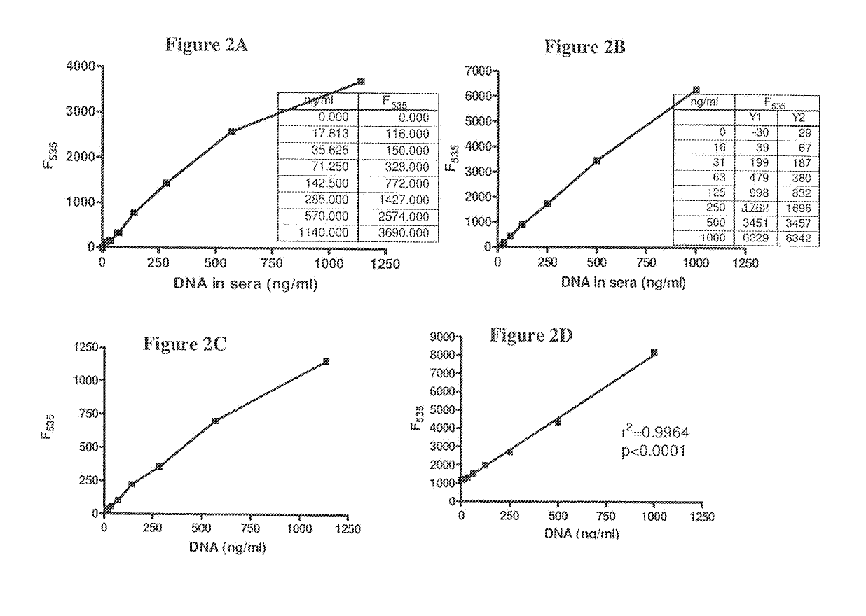

Similarly, fluorescence of side-by-side comparisons of serially diluted salmon DNA in 20% normal pooled human sera showed comparable detection, when two different intercalating agents were utilized (FIGS. 2A and 2B). Detection using SYBR gold in whole blood yielded comparable results (FIG. 2C) as did detection using EvaGreen (FIG. 2D).

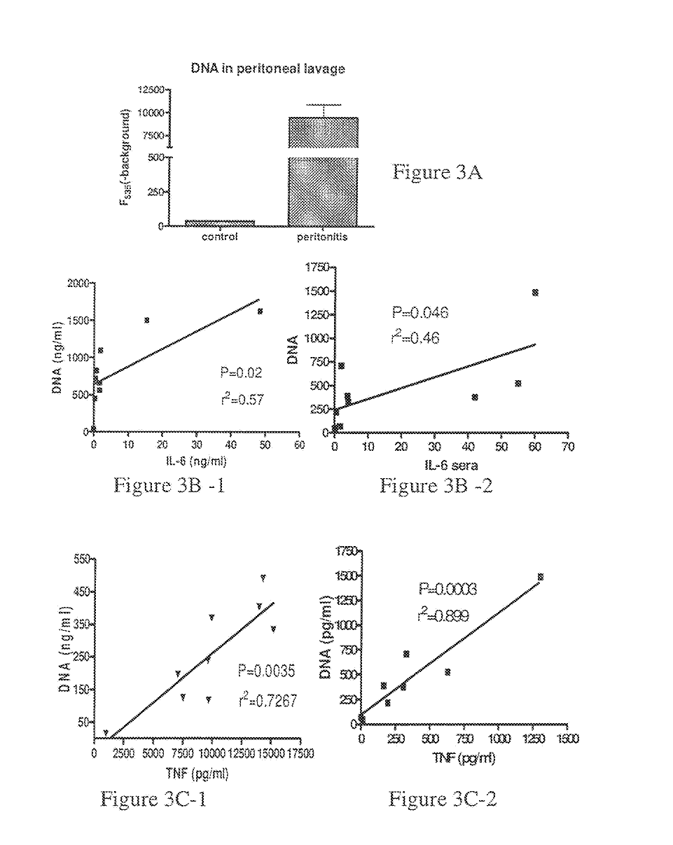

In order to determine whether rapid DNA quantification was obtainable in biological samples, peritoneal fluid obtained by lavage of mice undergoing peritonitis induced by intra-peritoneal E. coli injection was mixed with the intercalating agent, without prior DNA extraction (FIG. 3A). Mice undergoing E. coli-induced inflammatory responses demonstrated significantly greater amounts of free DNA in biological fluids as compared to controls.

Moreover, total DNA concentration correlated well with the presence of IL-6 and TNF induction in lavage fluid and in serum (FIGS. 3B and 3C, respectively).

### Example 2

### Rapid DNA Quantification in Human Sera

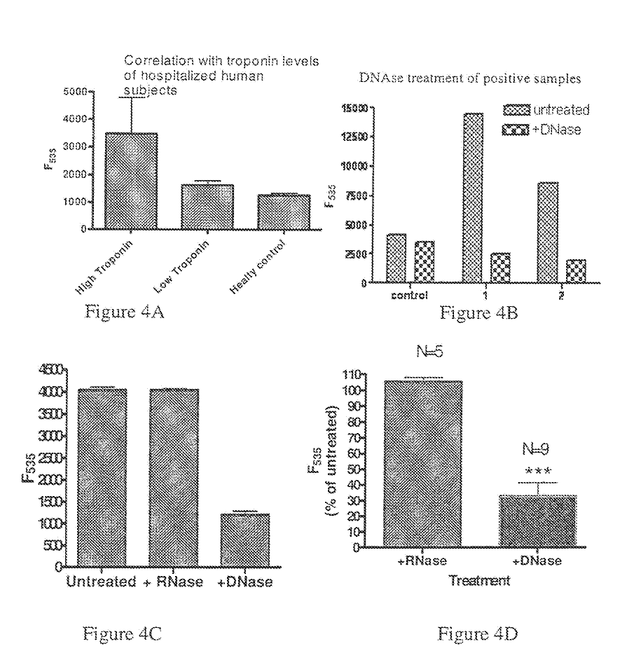

Example 1 demonstrated rapid DNA quantification in biological fluids including sera of mice, thus it was of interest to determine whether such assay would be useful as an indicator of DNA concentration in human sera. Toward this end, serum was collected from Human subjects arriving at the Emergency room with suspected myocardial infarction. FIG. 4A demonstrates that serum troponin levels (a protein released from cardiac muscle following an ischemic event) correlate well with DNA levels detected by the assay as described herein, again without necessity for DNA extraction prior to quantification. Treatment of the serum sample with DNase abolished detection indicating the specificity of the assay (FIG. 4B).

FIGS. 4C and 4D demonstrate the specificity of the assay for DNA and no other nucleic acid, as addition of RNase did not abrogate detection (FIGS. 4C and 4D). Patient samples were treated with RNase or DNase, and the percent fluorescence reduction of 5 different samples treated with RNase (n=5) and 9 with DNase were compared before (100%) and after treatment.

FIGS. 4E-G show quantification of DNA in samples from hospitalized patients with acute myocardial infarction (MI) at different hours from arrival to emergency room. FIGS. 4H-J depict the DNA levels (H), Distribution, (I) and patients outcome (J) of 200 subjects who were evaluated in a Hospital Emergency Room. A trend was evident that subjects who visited a Hospital Emergency Room had higher serum DNA levels as compared to healthy subjects (4H). FIG. 4J demonstrates the usefulness of the assay as a predictor for mortality, with an almost 50% mortality rate in subjects representing the upper 5% of subjects assay demonstrating high DNA concentration.

Thus a rapid, cost-effective and easy to use assay for DNA quantification in biological fluids has herein been developed, which does not necessitate prior DNA extraction.

### Example 3

### Rapid DNA Quantification in the Absence of Prior DNA Extraction

In order to delineate whether the sensitivity of detection is compromised without prior DNA extraction, side-by-side DNA quantification was conducted on samples in which DNA was subjected to a prior extraction step, or not (FIG. 5).

Panel A describes the dose-dependent fluorescence of DNA samples isolated from whole blood and extracted, per the QIAamp DNA blood Kit (Qiagen). DNA was extracted from healthy donor leukocytes, and suspended in buffer with a final concentration of 20% normal human serum, which does not appreciably differ from Panel C, showing direct DNA assay, without prior extraction.

Panel B describes the correlation of DNA samples isolated from whole blood and extracted, per the QIAamp DNA blood Kit (Qiagen) with β-globin copy number. Panel D shows the linear correlation of human DNA standards quantified in parallel by the conventional method and by SYBR gold.

Surprisingly, prior extraction of the DNA samples did not result in appreciably different results regarding DNA quantification, and moreover, detection may be somewhat compromised by prior extraction. These data support the fact that the rapid DNA quantification assay of this invention is highly specific, cost-effective, and non-labor intensive.

### Example 4

### Rapid DNA Quantification Assay Stability

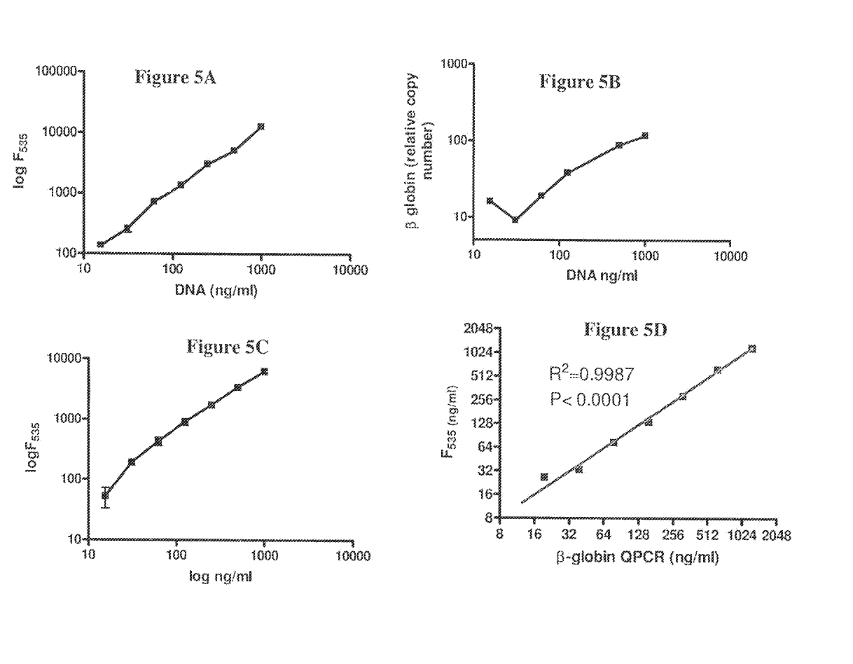

To determine whether test results were maintained stable over time, serum protein fluorescence and quenching was determined (FIG. 6). Pooled human serum was preincubated with DNase and diluted with PBS to various concentrations; same amount of salmon sperm DNA was added to all solutions resulting in a final concentration of 1140 ng/ml. Serum solutions at same concentrations not containing DNA were used to determine background fluorescence. A. Total and background fluorescence of serum solutions B. Calculated % quenching of the specific DNA signal [100−100×(Total F535−Background F535)/total F535]. Assay was performed in triplicates, ** indicates p<0.01 comparing serum solution to PBS without serum.

FIG. 6A demonstrates the sensitivity of the assay in detecting DNA concentration, in comparison to background fluorescence, and 6B indicates lack of appreciable quenching of the specific signal, with increasing serum concentrations, even up to serum levels of 30%.

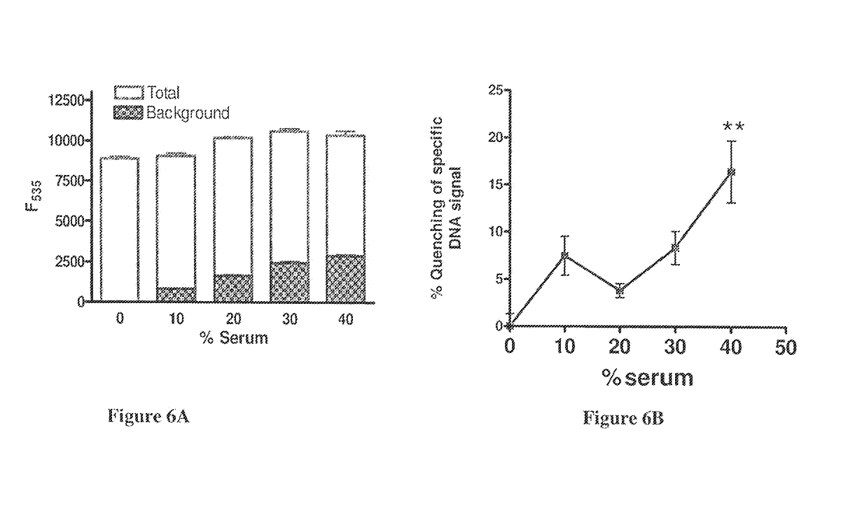

To determine whether the DNA in the test samples may be stable over time, whole blood samples were kept at room temperature or at 4° C. over time (FIG. 7A) with minimal differences observed in quantification of DNA for up to 6 hours in either case. Similarly, repeat freeze-thaw cycles (five) of sera did not appreciably alter DNA stability and thereby influence quantification, in comparison to samples at room temperature (FIG. 7B).

### Example 5

### Rapid DNA Quantification Assay Standardization

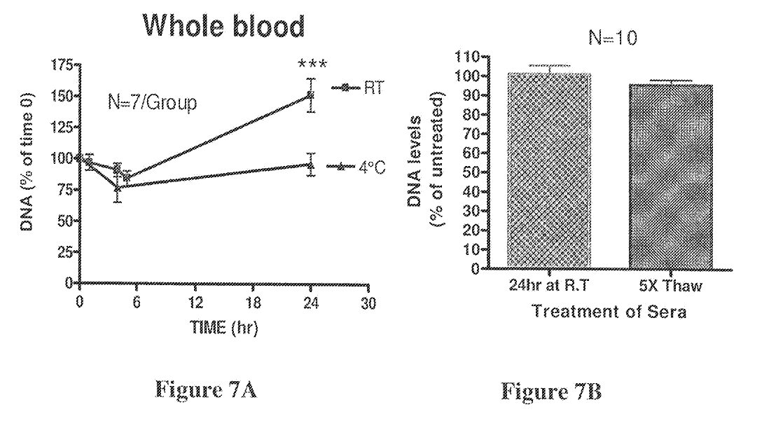

FIG. 8 demonstrates intra & inter assay variation. Intra-day and Intra-assay variation: To assess the variation of the assay, three patients sera were used containing normal, elevated and high DNA concentrations (197, 1096 and 4107 ng/ml, respectively) 8A. Intra-day variation of the assay was assessed by comparing readings of 12 assays of each sample in duplicates done independently on separate plates at different times over one working day. 8B. Day to day variation was assessed by comparing readings of 12 aliquots of each sample. Aliquots have been frozen and assayed on different days. For this assay, serum of three donors was used with low, elevated and high DNA concentrations (383, 1152 and 2735 ng/ml, respectively). Median value of the assays is indicated by the line inside the box. The Box indicates the distribution of 50% of the results and the bar above and below the box indicates 25% of the data.

When assay of the samples was repeated on different days, minimal variability between obtained results occurred, regardless of whether the DNA concentration in the serum was elevated or high (FIG. 8B). Some variation was observed, however, when samples containing a low DNA concentration were assayed. The cell free DNA assay range was evaluated in healthy donors of 47 consisted of 22 women and 25 man with an average age of 26.3±4.7 years. The volunteers were declared to be healthy and with no chronic disease. Subjects demonstrated some variability in terms of typical DNA concentrations and an average level of 471±203 ng/ml, was found. Thus the normal range (mean±2 std) is between 65-877 ng/ml in the samples tested.

### Example 6

### Rapid DNA Quantification as a Diagnostic and Prognostic Assay

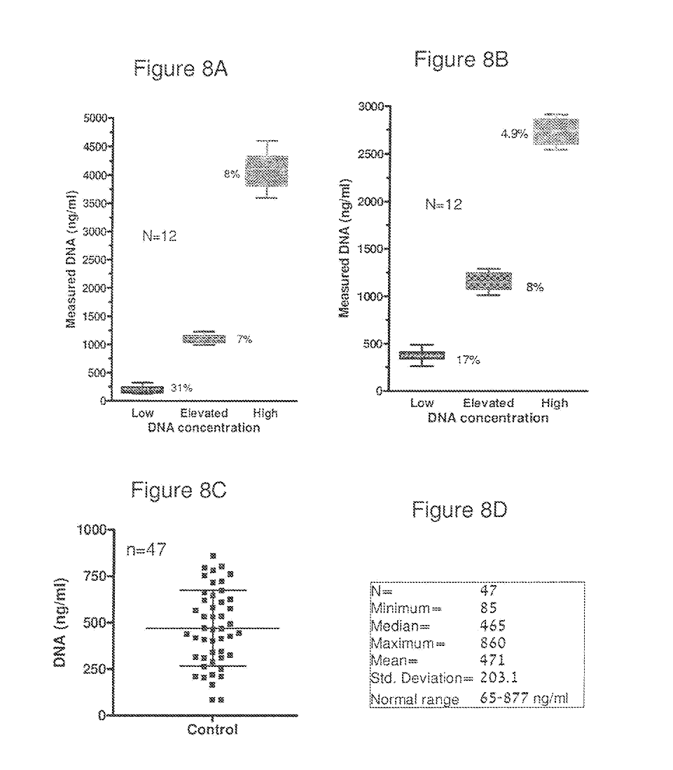

Example 2 demonstrates the potential usefulness of the rapid DNA quantification assays of this invention as a diagnostic and prognostic assay. To extend these studies, the assay was utilized to determine whether it can serve as an indicator of infection and potentially an indicator of severity of infection. FIG. 9A demonstrates that greater DNA concentration may be detected in serum collected from subjects with active EBV and CMV viral infection, as opposed to controls. FIG. 9B demonstrates that DNA quantification correlated well with viral load in HIV infected patients.

FIG. 9C demonstrates that in sepsis, as well, a clear increase in circulating DNA levels is observed, and that mortality correlated with highly elevated DNA levels. FIG. 9D demonstrated that in subjects with active peritonitis, leukocyte number in the peritoneum correlated well with DNA concentration.

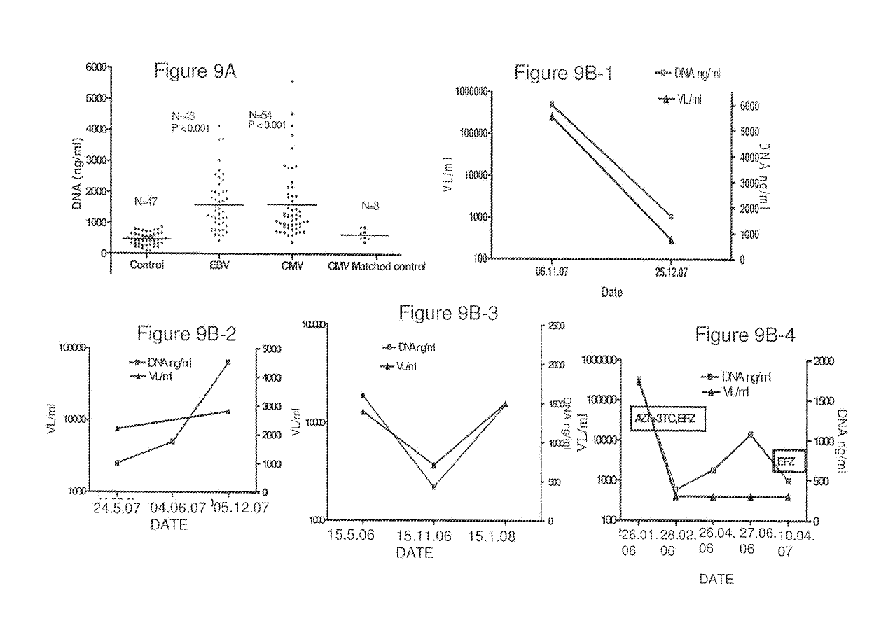

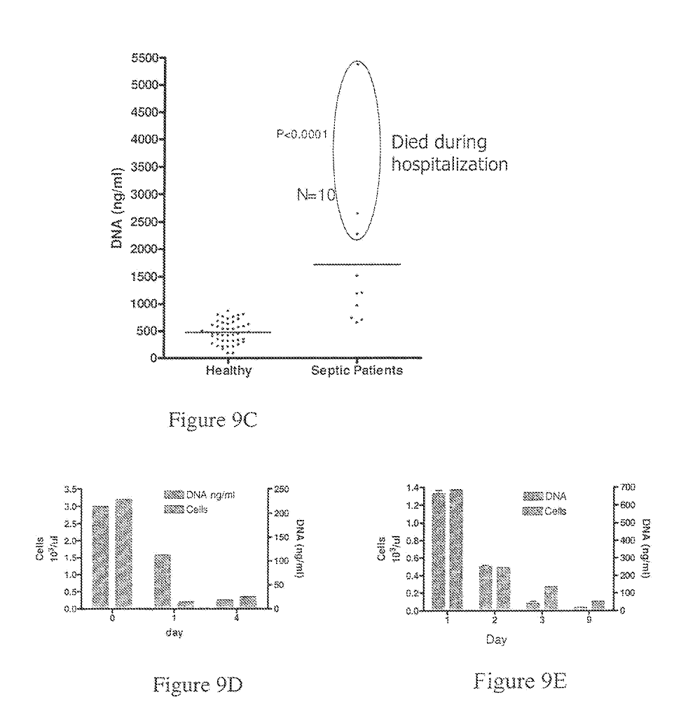

Other clinical conditions may similarly be identified via the use of the rapid DNA quantification assays of this invention. For example, recovery of grafted kidney function may be assessed, with DNA levels correlating well with creatinine levels, in patients following treatment with immunosuppressive drugs (FIG. 10). Circulating DNA levels in trauma patients rise as well (FIG. 11), with additional increases as a function of clinical complications arising in each subject, for example, the presence of pleural effusion in FIG. 11B, or subsequent bacteremia in FIG. 11E.

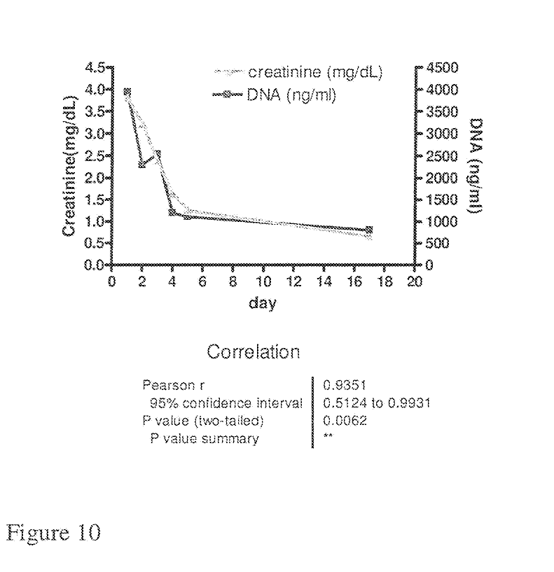

In order to determine whether detection of elevated DNA levels is found in cancer subjects, mice were inoculated intra footpad with an MCA-2 fibrosarcoma cell line with 1.0×10⁶ cells/mouse (N=10). Elevated circulating DNA levels were found to correlate with tumor size (FIG. 12B). CFD levels were elevated in patients with colon dancer, as well, one week before tumor removal (FIG. 12A).

### Example 7

### Rapid DNA Quantification as a Diagnostic and Prognostic Assay

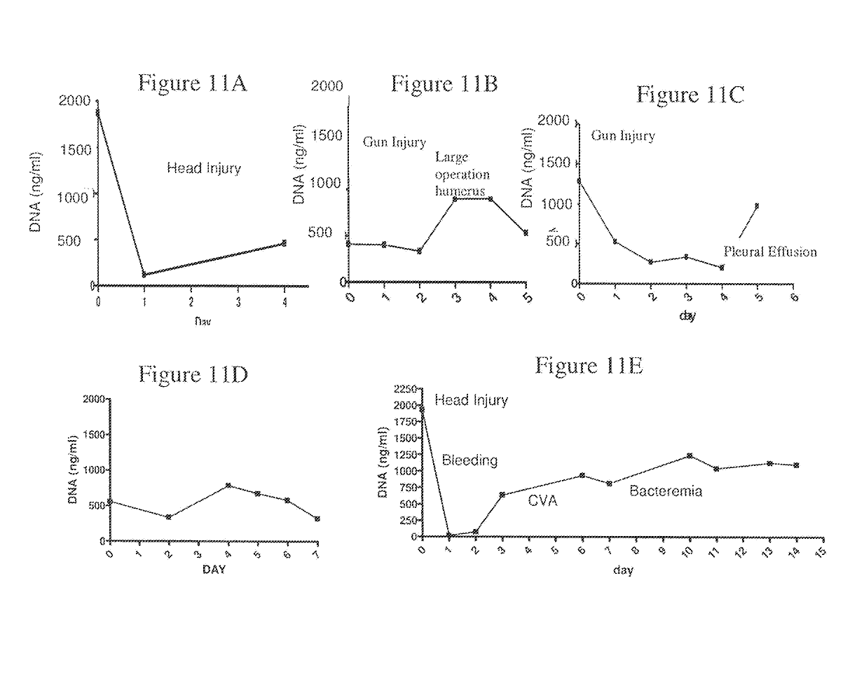

The assays of this invention may find use in tissue culture applications, as well. Rapid determination of DNA quantity in assays of cell lysates is not readily achievable. FIG. 13 demonstrates the measurement of DNA levels in cells lysates (0.1% NP40 in medium containing 10% FCS) and the linear relationship between DNA concentration and LDH activity detected. Supernatant free DNA quantification was determined (FIG. 13A) as was supernatant LDH activity (FIG. 13B), and the correlation between the two was plotted (FIG. 13C).

Thus, the assays of this invention provide for rapid DNA quantification, and determination of specific activities in cell lysates, providing a more quantitative analysis than has been achievable to date in other rapid assays.

### Example 8

### Rapid DNA Quantification as a Diagnostic and Prognostic Assay for Breast Cancer

### Materials and Methods

### Human Subjects

34 female patients with biopsy confirmed early-stage primary breast cancer before surgery were enrolled in the Soroka University Medical Center study, where women diagnosed with early stage breast cancer with surgery as the first treatment modality were included in the study and women with other malignancies and severe acute or chronic illness were excluded.

The control reference values were established by analysis of sera from 18 healthy women volunteers, from the small community village “Kibutz Mashabe Sade”. These volunteers joined the study following local advertisement. Their health condition was certified by their family physician which relied on interrogations and their medical records. Volunteers with acute or chronic disease were excluded. In addition we included in the study 9 early-stage primary breast cancer patients after surgery just before they receive their first radiation therapy (RT) treatment. Prior to RT, one patient was treated with Doxorubicin and Cyclophosphamide, two were treated with Doxorubicin, Cyclophosphamide and Paclitaxel and six patients were not pretreated. The Age, cancer stage and detected receptors are detailed in Table 1.

Blood samples (3 ml) were collected from the patients and the volunteers using BD Vacutainer® gel tubes (Becton-Dickinson, Plymouth, UK). For CFD levels determination serum was separated from the cellular fraction and frozen at −20° C. until assayed.

The research protocol was approved by the local ethics committee of the Soroka Medical Center, Beer-Sheva, Israel, and written informed consent was obtained from all patients and volunteers included in this study.

CFD assay

CFD was detected directly in sera according to the method recited hereinabove. Briefly; SYBR4® Gold Nucleic Acid Gel Stain, (Invitrogen, Paisley, UK) was diluted first at 1:1000 in Dimethyl sulfoxide (DMSO, Sigma-Aldrich, Rehovot, Israel) and then at 1:8 in PBS. Eight DNA standards (0 ng/ml and 7 serial dilutions from 78 ng/ml to 5000 ng/ml) were prepared with commercial Salmon sperm DNA (Sigma-Aldrich) in PBS containing 10% bovine serum albumin (BSA, Sigma-Aldrich). 10 μL of sera or DNA standard solutions were applied in duplicates to black 96 well plates (Greiner Bio-One, Frickenhausen, Germany). 40 μL of diluted SYBR® Gold was added to each well (final dilution 1:10,000) and fluorescence was measured with a 96 well fluorometer (Spectrafluor Plus, Tecan, Durham, N.C.) at an emission wavelength of 535 nm and an excitation wavelength of 485 nm. Concentrations of unknown samples were calculated from standards curve by extrapolation in a linear regression model. Usually, the “goodness of fit” of the standards curve (r2) was higher than 0.97. This method was tested in comparison with the gold standard, QPCR, and was found to be in good correlation of R2=0.9987 (p<0.0.0001) as previously described.

Statistical analysis was performed with GraphPad Prism® software (edition 5.01), statistically significant differences between two groups were tested the non-parametric Mann Whitney test. For comparison of more than two groups we used non-parametric Kruskal-Wallis test followed by Dunn's Multiple Comparison test. Correlation was analyzed by Spearman test. Results are given as median.

### Results

Table 1 depicts clinical and demographic characteristics of the study patients.

TABLE 1

| | Control | Pre-surgery | Post-surgery |
|---|---|---|---|
| N | 18 | 34 | 9 |
| Age (mean ± STD) | 49 ± 16.4 years | 63 ± 13.3 years | 62 ± 6.7 years |
| CA-15.3 | — | 17.2 U/ml | 21.7 U/ml |
| Stage | 0 | 1 (3%) | 2 (22%) |
| | 1 | 18 (53%) | 4 (44%) |
| | 2A | 6 (17%) | 1 (11%) |
| | 2B | 5 (15%) | 2 (22%) |
| | 3A | 4 (12%) | — |
| Receptors | ER− PR− Her− | 5 (15%) | 2 (22%) |
| | ER− PR− Her+ | 1 (3%) | — |
| | ER+ PR− Her− | 8 (24%) | — |
| | ER+ PR+ Her− | 20 (59%) | 6 (67%) |
| | ER+ PR+ Her+ | — | 1 (11%) |

The patients in the Pre-surgery group were older than those in the Control group (63±13.3 vs. 49±16.4 years). There were no age and baseline CA-15.3 levels (17.2 vs. 21.7 U/ml) differences between the pre- and post-surgery groups (63±13.3 vs. 62±6.7 years). The distribution between stages in pre- and post-surgery were comparable, the largest stage group was stage-1. In both groups most of the patients' hormone receptors were ER+, PR+, Her−.

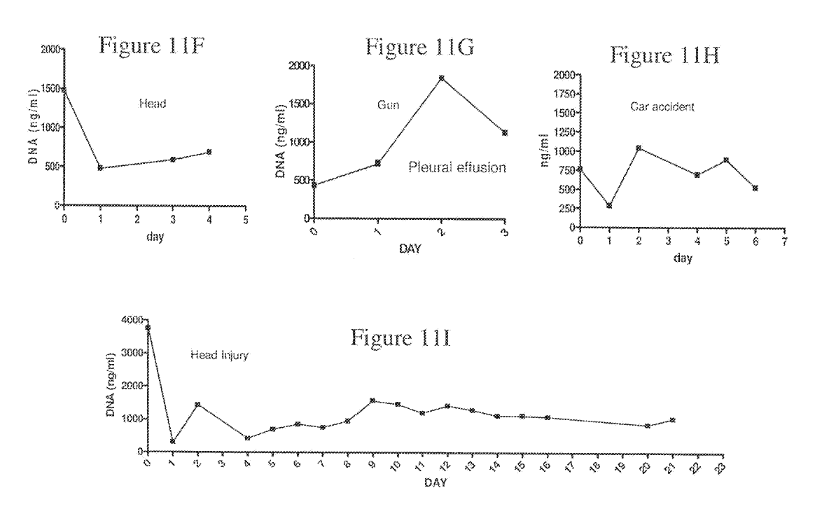

As shown in FIG. 14A, pre-surgery breast cancer patients had elevated CFD levels of 888 ng/ml (95% CI, 783-1254) significantly higher (p<0.001) than healthy control group with a value of 372 ng/ml, (95% CI, 278-510). The post-surgery group had CFD levels of 352 ng/ml (95% CI, 274-522) lower than pre-surgery group (p<0.01) and similar to the control group.

Receiver operating characteristic (ROC) analysis of the CFD levels of the pre-surgery cancer patient and healthy group demonstrated an area under the curve (AUC) of 0.81 (95% CI, 0.69 to 0.93) (FIG. 14B). Based on previous studies a cutoff levels of 850 ng/ml was set. At this value the calculated sensitivity for detection of early-stage breast cancer in the cohort was 51.5% (95% CI, 34-66%) and the specificity 100% (95% CI, 78-100%).

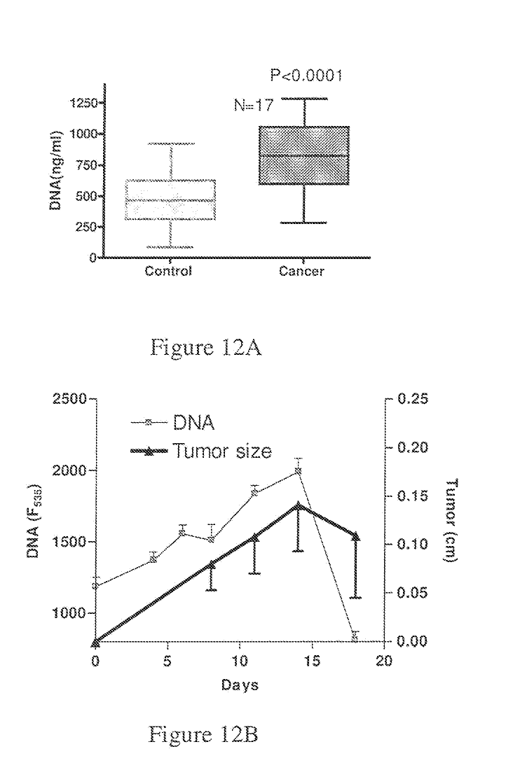

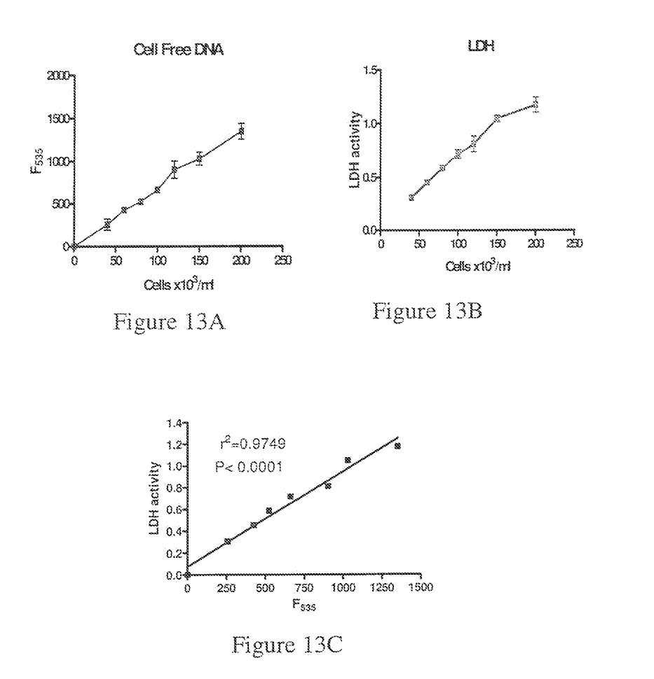

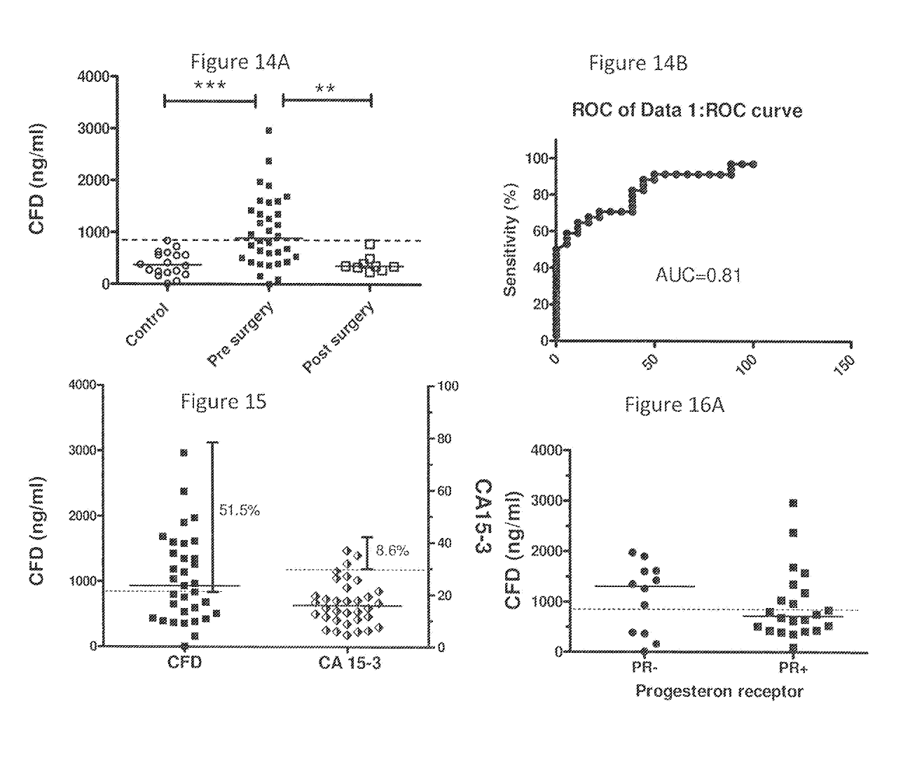

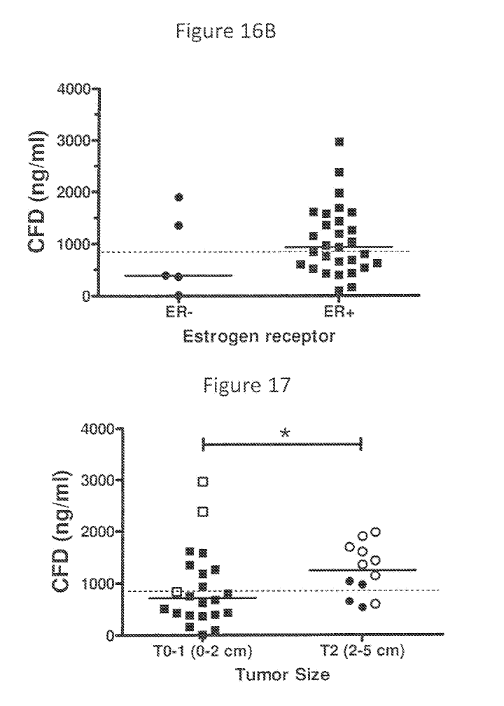

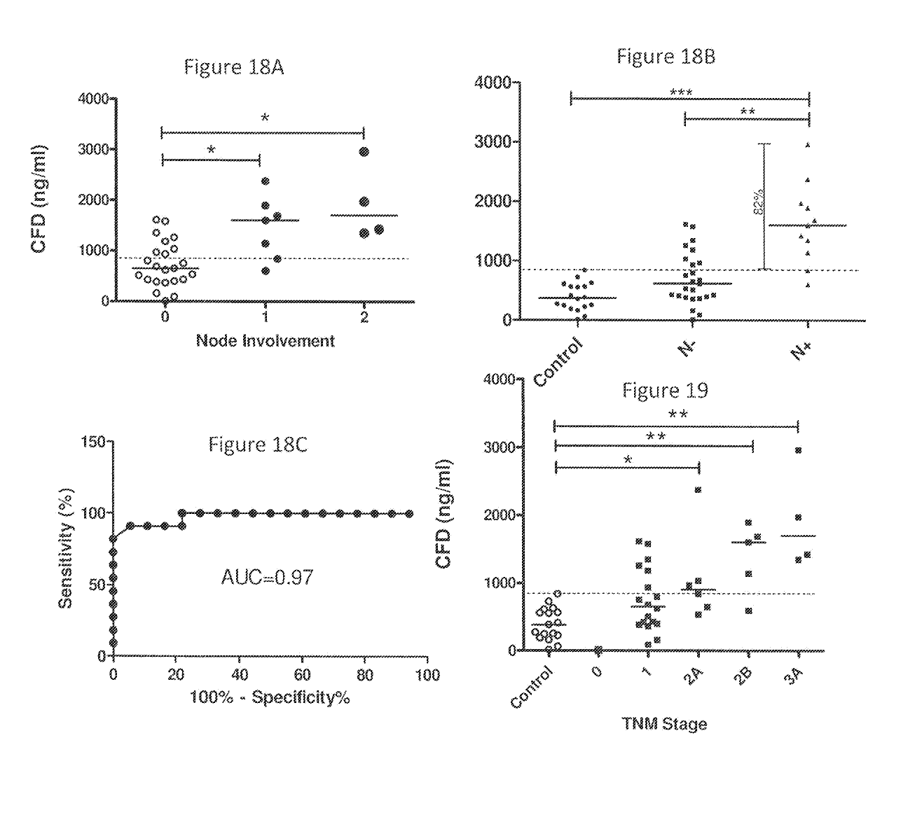

To evaluate the biomarker positivity by the assay as herein described was compared to CA-15.3 positivity, which is the gold standard biomarker for follow-up of breast cancer recurrence. As shown in FIG. 15 only 8.6% were slightly positive to CA15.3 while 51.5% of the pre-surgery breast cancer patients were CFD positive. Correlating CFD to age, CA15.3, Histology tumor typing and grade, tumor size, nodal involvement and TNM stage was undertaken, as well. Correlations to tumor size (p=0.02, Rho=0.39), nodal involvement (p=0.0001, Rho=0.62) and TNM stage (p=0.0001, Rho=0.62) were found. No correlation was found between CFD and age, CA15.3 or to histology. As shown in FIG. 16, the presence of hormone receptors had no effect on CFD levels. In contrast, CFD levels were elevated in patients with larger tumor (FIG. 17) and lymph nodes involvement (FIG. 18). The sensitivity to detect cancer patients with nodal involvement (N+) was elevated (82%, FIG. 18B) and the ROC curve (FIG. 18C) was almost perfect with AUC of 0.97 (95% CI, 0.93 to 1.02) and not related to tumor size. Even small tumors (T0-1) with lymph node involvement had relatively high CFD levels (FIG. 19). The sensitivity of CFD increases according to TNM stage. As shown in FIG. 19, 30% of the patients in stage 1, 50% in 2A, 80% in 2B and 100% of the patients in stage 3A were CDF positive.

The simple technique to measure CFD levels was applied as described hereinabove, i.e., DNA was measured directly in the diluted samples using a fluorescent dye and elevated CFD levels in breast cancer patients before surgery were found, while normal levels were found in post-surgery patients. CFD levels in breast cancer patients were compared to CA15.3 positivity. The CFD assay had a sensitivity of 51.5%, to detect patients with early stage cancer, which was higher than the sensitivity of CA15.3 (8.6%).

Furthermore, CFD levels correlated with tumor size. Moreover, the assay was highly sensitive (82%) for detection of women with lymph nodes involvement (ROC AUC=0.97).

Tumor stage was in positive correlation with CFD levels and the percent of CFD positive samples gradually increased from 30% for patients at stage 1 to 100% for stage 3A. This gradual increase in sensitivity means that patients that were tested negative at a very early stage have the chance to be detected in more advance stage while the disease is still curable.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method of assessing breast cancer risk, said method comprising quantifying the DNA concentration in a blood sample from a subject, the method comprising:
mixing a blood sample with a fluorescent nucleic acid intercalating agent, wherein said mixing is conducted in the absence of prior nucleic acid extraction;
detecting fluorescence of said fluorescent nucleic acid intercalating agent; and
correlating detection of said fluorescence with a value, wherein if said value represents an increased concentration of nucleic acid in said biological fluid sample versus that of a standard sample then said subject has an elevated risk for breast cancer.

2. The method of claim 1, wherein said detecting is conducted with the use of a fluorimeter.

3. The method of claim 1, wherein said detectable nucleic acid intercalating agent comprises SYBR Gold® or SYBR Green®.

4. The method of claim 1, wherein said biological fluid is whole blood.

5. The method of claim 1, wherein said biological fluid is serum or plasma.

6. The method of claim 1, wherein said method further comprises correlating results in said value with a predictive value indicative of related lymph node neoplasia.

7. The method of claim 1, wherein said method further comprises correlating results in said value with a predictive value indicative of cancer stage in said subject.

8. The method of claim 1, wherein if said value exceeds 850 ng/ml, then said method further comprises providing a recommendation for mammography of said subject.

9. The method of claim 1, wherein if said value exceeds 1400 ng/ml, then said method further comprises providing a recommendation to avoid removal of proximal lymph nodes in said subject.

10. A method of assessing breast cancer severity in a subject, said method comprising quantifying the DNA concentration in a blood sample from a subject, the method comprising:
mixing a blood sample with a fluorescent nucleic acid intercalating agent, wherein said mixing is conducted in the absence of prior nucleic acid extraction;
detecting fluorescence of said fluorescent nucleic acid intercalating agent; and
correlating detection of said fluorescence with a value, wherein if said value represents an increased concentration of nucleic acid in said biological fluid sample versus that of a standard sample then said subject has breast cancer of an advanced stage.

11. The method of claim 10, wherein said detecting is conducted with the use of a fluorimeter.

12. The method of claim 10, wherein said detectable nucleic acid intercalating agent comprises SYBR Gold© or SYBR Green©.

13. The method of claim 10, wherein said biological fluid is whole blood.

14. The method of claim 10, wherein said biological fluid is serum or plasma.

15. A method of assessing responsiveness to a breast cancer treatment modality in a subject, said method comprising quantifying the DNA concentration in a blood sample from a subject, the method comprising:
mixing a blood sample with a fluorescent nucleic acid intercalating agent, wherein said mixing is conducted in the absence of prior nucleic acid extraction;
detecting fluorescence of said fluorescent nucleic acid intercalating agent; and
correlating detection of said fluorescence with a value, wherein if said value represents an increased concentration of nucleic acid in said biological fluid sample versus that of a standard sample then a responsiveness of said subject to a breast cancer treatment modality is poor.

16. The method of claim 15, wherein said correlating detection of said fluorescence with a value is with a value representing a baseline determination taken from said subject prior to commencement of said treatment modality.

17. The method of claim 16, wherein said correlating is against a value obtained for a standard sample and said baseline determination.

18.-24. (canceled)


SEQUENCE LISTING (2 sequences)

SEQ ID NO: 1 - DNA, 20 bp, Artificial Sequence
  synthetic human beta-globin gene quantitative real-time PCR (QPCR) amplification forward primer
acacaactgtgttcactagc

SEQ ID NO: 2 - DNA, 20 bp, Artificial Sequence
  synthetic human beta-globin gene quantitative real-time PCR (QPCR) amplification reverse primer
caacttcatccacgttcacc